(19) United States
(12) Patent Application Publication
(10) Pub. No.: US 2026/0008814 A1
(43) Pub. Date: Jan. 8, 2026

(54) DEVELOPMENT OF PEPTIDES WITH ANTI-CANCER AND ANTIMICROBIAL PROPERTIES

(71) Applicants: YILDIZ TEKNIK ÜNIVERSITESI DÖNER SERMAYE ISLETME MÜD, Esenler/Istanbul (TR); YILDIZ TEKNOLOJI TRANSFER OFISI ANONIM SIRKETI, Esenler/Istanbul (TR)

(72) Inventors: Sümeyra AYAN, Esenler/Istanbul (TR); Selcen ARI YUKA, Esenler/Istanbul (TR); Alper YILMAZ, Esenler/Istanbul (TR); Cem Bülent ÜSTÜNDAG, Esenler/Istanbul (TR)

(21) Appl. No.: 18/880,355

(22) PCT Filed: Mar. 8, 2024
(86) PCT No.: PCT/TR2024/050228

§ 371 Date: Dec. 31, 2024

(30) Foreign Application Priority Data
Mar. 20, 2023 (TR) 2023/003032

(51) Int. Cl. C07K 7/08; G01N 33/68

(52) U.S. Cl. CPC C07K 7/08; G01N 33/6845

(57) ABSTRACT

Peptides for use in systems that prevent the interaction of the FadA protein released from the Fusobacterium nucleatum (F. nucleatum) bacterium with E-cadherin, which has a carcinogenic effect, or the inhibition of the microorganism with antimicrobial agents and that will inhibit the carcinogenesis mechanisms of F. nucleatum infection and enable the development of therapeutic systems against infections that may occur due to the decreased immunity of patients undergoing cancer treatment with the use of peptides with anti-cancer and antimicrobial properties by developing ten peptides with FadA protein binding energy greater than −11.6 kcal/mol. Peptides with anticancer and antimicrobial properties allow the development of therapeutic systems to prevent F. nucleatum infection, to prevent cancer development after F. nucleatum infection, or to develop therapeutic systems against infections that occur due to the decreased immunity of cancer patients due to cancer treatment.

### TECHNICAL FIELD

The invention relates to peptides that prevents the interaction of the FadA protein released from the Fusobacterium nucleatum (F. nucleatum) bacterium with E-cadherin, which has a carcinogenic effect, or the inhibition of the microorganism with antimicrobial agents and that will inhibit the carcinogenesis mechanisms of F. nucleatum infection and enable the development of therapeutic systems against infections that may occur due to the decreased immunity of patients undergoing cancer treatment with the use of peptides with anti-cancer and antimicrobial properties by developing ten peptides with FadA protein binding energy greater than −11.6 kcal/mol.

### PRIOR ART

Although methods such as surgical interventions, radiotherapy, chemotherapy and immunotherapy are used for the treatment of colorectal cancer, which is one of the most common types of cancer in the world today, there is no definitive treatment method yet. Colorectal cancer develops under the multilayered influence of environmental, nutritional and genetic factors. Since the microbiota of the gastrointestinal (GI) system is quite rich, the roles of microorganisms in the mechanisms of diseases in this system should not be ignored. Gram-negative non-sporulating F. nucleatum is effective in the development of oral infections, GI diseases, cardiovascular diseases and many other infectious diseases. The results of metagenomic sequencing studies have shown that there may be a significant relationship between F. nucleatum and colorectal cancer. Although F. nucleatum is seen to be abundant in colorectal cancer patients, it has been reported that the presence of the bacterium results in a poor prognosis due to chemotherapy resistance. It is known that F. nucleatum exerts its effect on colorectal cancer by activating the WNT/β-catenin pathway through E-cadherin interaction, mediated by the FadA protein. The use of broad- and narrow-spectrum antibiotics has been recommended to prevent the role of F. nucleatum in the aggressive course in colorectal cancer, but antibiotic resistance has revealed the problem that the carcinogenesis effect may be more aggressive. Therefore, approaches that can block bacteria-cell interactions in a receptor-specific manner may equalise the role of F. nucleatum in colorectal cancer. Infection is one of the most common complications in cancer patients. It is stated in the literature that in 2008, approximately 2 million new cancer cases worldwide were associated with infection. If these infections can be prevented and/or treated, it is predicted that there will be fewer cases of cancer, including in less developed parts of the world. However, the high use of antibiotics against infection in treatment causes the microorganism to become resistant to the antibiotic used for treatment over time. If the infection occurs again, treatment processes that result in the death of the patient are observed, as the antibiotic-resistant microorganism cannot be treated. One of the most important problems in the development of antibacterial agents is overcoming the antibiotic resistance mechanisms of bacteria. The most promising approach to overcome the resistance mechanisms developed against antibacterial agents is to disrupt the integrity of bacterial cell walls with cationic agents. Cationic peptides stand out in this field as one of the most potential candidates for biocompatible and stable antibacterial applications. Silver (Ag), which has the highest electrical conductivity among all metals, is one of the products used as cationic agents. Ag, a soft, ductile and malleable metal, is chemically inactive, stable in water and does not oxidise in air. Silver nanoparticles (AgNPs), with their unique physicochemical properties (small size, larger surface area, surface chemistry, shape, particle morphology, particle composition, coating/capping, agglomeration, particle dissolution rate, etc.) are produced every year for use in various applications in the nanoparticle (NP) form due to their antimicrobial, optical, electrical and catalytic properties. Many NPs show activity through ionic interactions rather than receptor-specific interactions. This causes off-target side effects of NPs. Concerns about the possible side effects of AgNPs on the environment and human health are also increasing at the same rate. After AgNPs are administered to the body, approximately 30% to 99% of the nano-silver dose accumulates in the liver. This leads to a decrease in the ability of AgNPs used as agents to deliver the target chemical to diseased tissues and potentially an increase in toxicity at the hepatocyte level.

With all this, therapeutic peptides are more promising in this field. Therapeutic peptides are amino acid sequences that can activate or suppress specific molecular pathways through protein interactions. Peptides attract attention in therapeutic applications due to their flexible interaction ability, biocompatibility and ability to be designed according to the operating conditions in which they can be functional.

In the article in the state of the art by Rubinstein M R, Wang X, Liu W, Hao Y, Cai G, and Han Y W titled ‘Fusobacterium nucleatum promotes colorectal carcinogenesis by modulating E-cadherin/β-catenin signaling via its FadA adhesin’ on promotion of colorectal carcinogenesis by F. nucleatum by modulating E-cadherin/β-catenin signaling through the adhesiveness of FadA protein published in Cell Host Microbe journal in 2013, testing the role of EC5 in F. nucleatum attachment and invasion, as the FadA protein is bound to the EC5 domain of E-cadherin, was studied. F. nucleatum attachment and invasion of HCT116 were inhibited by purified GST-EC5 fusion protein in a dose-dependent manner, with maximum inhibition observed at 0.1 μM. Synthetic peptides derived from different regions of EC5 were then tested for their ability to inhibit F. nucleatum attachment and invasion. Peptide 3 corresponding to region 3 exhibited similar inhibitory effect as EC5. Sequential deletions of peptide 3 were generated to determine the minimal sequences required for inhibition. The results showed that the peptide containing 11 amino acids, the sequence ASANWTIQYND, was the required minimum and was the inhibitory peptide (IP). The ASANWTIQYND peptide sequence is a sequence obtained from the CDH1 protein region that interacts with the bacterial FadA protein. This sequence mimics CDH1 and provides its therapeutic efficacy. When ASANWTIQYND:FadA interactions and CDH1:FadA interactions were subjected to molecular docking analysis, no significant difference was observed in binding energy. This is an indication that the effectiveness of the ASANWTIQYND reference sequence, which is effective in in vitro conditions, may decrease in a three-dimensional (3D) complex tissue environment. At the same time, if bacterial FadA protein production continues after the degradation of the peptide under physiological conditions, it poses the risk of the presence of CDH1 stimuli.

In the prior art document numbered US2014206011A1, methods and compositions of diagnosis and treatment of disorders related to F. nucleatum are described. It describes a method for identifying a subject at high risk of colorectal cancer. The method for identifying a subject at high risk of colorectal cancer comprises taking a biological sample from the subject, measuring the level of F. nucleatum in the biological sample, and comparing the measured level to a control level. The embodiments described therein relate to a method for identifying a subject at high risk of colorectal cancer. The method comprises collecting a biological sample from the subject. The level of F. nucleatum in the biological sample is measured. The measured level is compared with a control level. It relates to a method of inhibiting F. nucleatum colonisation in a subject. The method comprises administering a therapeutically effective amount of a therapeutic agent that prevents F. nucleatum from binding to or complexing with E-cadherin. The therapeutic polypeptide, consisting of approximately 10 to 50 amino acids, has been shown to bind to the FadA protein and has at least 80% sequence identity with approximately 10 to 50 consecutive amino acids of E-cadherin. Herein, the therapeutic polypeptide has the amino acid sequence ASANWTIQYN (SEQ ID NO: 1) or NNFTLTDNHDN (SEQ ID NO: 2). The binding energy of the reference peptide SEQ ID NO: 1 used here to the FadA protein is −11.6 kcal/mol.

Although the ASANWTIQYND peptide sequence in the state of the art has an amino acid sequence that is frequently used in this field, there are difficulties in half-life and water solubility under physiological conditions. For this reason, it is necessary to determine more than one peptide sequence that will exhibit different behavioural profiles under physiological conditions. Therefore, it is necessary to carry out R&D studies to determine more peptide sequences by performing in silico studies. Peptides with anticancer and antimicrobial properties will allow the development of therapeutic systems to prevent F. nucleatum infection, to prevent cancer development after F. nucleatum infection, or to develop therapeutic systems against infections that may occur due to the decreased immunity of cancer patients due to cancer treatment, by making end modifications, conjugating with other molecules, using it as a coating agent in other treatment applications, or integrating it into a micro/nano-sized carrier system. In particular, it is anticipated that the peptides to be developed should contain peptide sequences with the highest binding energy to the FadA protein compared to the ASANWTIQYND sequence.

Therefore, there is a need to develop new peptide-based technologies. It is possible to develop therapeutic peptides targeted to molecular interactions in disease-specific biological processes.

### The Aim of the Invention

The main aim of the invention is to develop ten peptide sequences with FadA protein binding energy greater than −11.6 kcal/mol, end modifications of which with anticancer and antimicrobial properties can be made, that can be conjugated with other molecules, used as a coating agent in other treatment applications, or integrated into micro/nano-sized carrier systems, preventing the interaction of the FadA protein released from the Fusobacterium nucleatum (F. Nucleatum) bacterium with E-cadherin, which has a carcinogenic effect, or inhibiting the microorganism with antimicrobial agents.

Another aim of the invention is to develop ten peptide sequences to develop a therapeutic system against infections that may occur due to the decreased immunity of cancer patients undergoing cancer treatment.

The aim of the invention is to provide the first ten peptide sequences that are greater in binding energy than that of the ‘ASANWTIQYND’ peptide sequence used as a reference to the FadA protein.

Another aim of the invention is to develop biocompatible peptide sequences with flexible interaction capabilities that can activate or suppress specific molecular pathways through protein interactions.

The aim of the invention is to develop TYYLRRTYKKQRH (sequence ID number 1), YRNWTIQRYRILR (sequence ID number 2), LRLIRRTIQVRTR (sequence ID number 3), RYYYLNWTIQRLR (sequence ID number 4), LRYRYNTIQYALR (sequence ID number 5), RRNWTWQRRLLRR (sequence ID number 6), YRRLRRLYRWYRY (sequence ID number 7), RYYRNWYNYYRWY (sequence ID number 8), YYLYRNNWLIQLR (sequence ID number 9) and RLLREWLNWTIQR (sequence ID number 10) peptides that have the ability to bind to the FadA protein with high affinity.

Another aim of the invention is to obtain sequence ID number 1, sequence ID number 2, sequence ID number 3, sequence ID number 4, sequence ID number 5, sequence ID number 6, sequence ID number 7, sequence ID number 8, sequence ID number 9 and sequence ID number 10 peptide sequences with higher than CDH1 binding energy.

The aim of the invention is to obtain sequence ID number 1, sequence ID number 2, sequence ID number 3, sequence ID number 4, sequence ID number 5, sequence ID number 6, sequence ID number 7, sequence ID number 8, sequence ID number 9 and sequence ID number 10 peptide sequences that inhibit tumorigenesis mechanisms as a result of molecular docking and antimicrobial activity analyses by selecting 2000 peptides, 13 amino acids long, randomly selected from all amino acids, 5000 peptides consisting of 8 amino acids of LYR in a random sequence and in which the NWTIQ motif is randomly positioned [asparagine (Asn, N), tryptophan (Trp, W), threonine (Thr, T), isoleucine (Ile, 1) and glutamine (Gln, Q)] and 3000 peptides with a random sequence of 8 amino acids from all amino acids and in which the NWTIQ motif is randomly positioned since the amino acids leucine (Leu, L), arginine (Arg, R) and tyrosine (Tyr, Y) are especially effective in the intra-interactions of the FadA-FadA protein.

By local molecular docking analysis of randomly derived peptide sequences, binding energies (kcal/mol) are estimated by positioning the peptide sequences provided as input files in affinity maps defined in local binding sites using ten thousand random sequences AutoDock CrankPep (ADCP) and 4973 peptide sequences showing the best affinity (binding affinity<−14.0 kcal/mol) are selected, then by expanding high-affinity random sequences with a genetic algorithm and local molecular docking analysis of the sequences, the peptide sequences selected in the previous step are multiplied by a genetic algorithm and mutation principle written in the R program and 32637 new sequences produced from 4973 best binding peptides are analysed under the same conditions as ADCP, then the 37610 peptides for which molecular docking analysis was performed are sorted according to their binding affinity, and the 100 sequences showing the best binding energy (the most negative) are compiled as those that meet with the amp_probability>0.5 condition and as a result of AMP prediction, 56 out of 100 peptides are selected because they meet with the >0.5 condition in all algorithms [Random Forest (RF), Support Vector Machine (SVM) and Artificial Neural Networks (ANN)]. The interactions that flexible peptide structures can have with the FadA protein are analysed with both local (ADCP) and global (HPEPDOCK) molecular docking programs, and then the evaluation of physical and chemical properties of peptides according to physiological conditions is carried out with the ProtParam tool, and as a result, after molecular docking and antimicrobial activity analysis, sequence ID number 1, sequence ID number 2, sequence ID number 3, sequence ID number 4, sequence ID number 5, sequence ID number 6, sequence ID number 7, sequence ID number 8, sequence ID number 9, sequence ID number 10, which are peptides that may show tumorigenesis inhibitor and antimicrobial activity, are obtained.

The subject of the invention is having TYYLRRTYKKQRH (sequence ID number 1) or YRNWTIQRYRILR (sequence ID number 2) or LRLIRRTIQVRTR (sequence ID number 3) or RYYYLNWTIQRLR (sequence ID number 4) or LRYRYNTIQYALR (sequence ID number 5) or RRNWTWQRRLLRR (sequence ID number 6) or YRRLRRLYRWYRY (sequence ID number 7) or RYYRNWYNYYRWY (sequence ID number 8) or YYLYRNNWLIQLR (sequence ID number 9) or RLLREWLNWTIQR (sequence ID number 10 peptide sequences that show tumorigenesis inhibitor and antimicrobial activity after molecular docking and antimicrobial activity analysis.

The subject of the invention is a peptide that shows tumorigenesis inhibitor and antimicrobial activity after molecular docking and antimicrobial activity analysis, wherein
TYYLRRTYKKQRH (sequence ID number 1) peptide comprises 23.1% arginine (Arg) (R) and 7.7% glutamine (Gln) (Q), 7.7% histidine (His) (H), 7.7% Leucine (Leu) (L), 15.4% lysine (Lys) (K), 15.4% threonine Thr (T), and 23.1% tyrosine Tyr (Y) by weight,
YRNWTIQRYRILR (sequence ID number 2) peptide comprises 30.8% Arg (R) and 7.7% asparagine (Asn) (N), 7.7% Gln (Q), 15.4% isoleucine (Ile) (1), 7.7% Leu (L), 7.7% Thr (T), 7.7% tryptophan (Trp) (W), and 15.4% Tyr (Y) by weight,
LRLIRRTIQVRTR (sequence ID number 3) peptide comprises 38.5% Arg (R), 7.7% Gln (Q), 15.4% Ile (1), 15.4% Leu (L), 15.4% Thr (T), and 7.7% valine (Val) (V) by weight,
RYYYLNWTIQRLR (sequence ID number 4) peptide comprises 23.1% Arg (R), 7.7% Asn (N), 7.7% Gln (Q), 7.7% Ile (1), 15.4% Leu (L), 7.7% Thr (T), 7.7% Trp (W), 23.1% Tyr (Y) by weight,
LRYRYNTIQYALR (sequence ID number 5) peptide comprises 7.7% alanine (Ala) (A), 23.1% Arg (R), 7.7% Asn (N), 7.7% Gln (Q), 7.7% Ile (1), 15.4% Leu (L), 7.7% Thr (T), 23.1% Tyr (Y) by weight,
RRNWTWQRRLLRR (sequence ID number 6) peptide comprises 46.2% Arg (R), 7.7% Asn (N), 7.7% Gln (Q), 15.4% Leu (L), 7.7% Thr (T), 15.4% Trp (W) by weight,
YRRLRRLYRWYRY (sequence ID number 7) peptide comprises 46.2% Arg (R), 15.4% Leu (L), 7.7% Trp (W), 30.8% Tyr (Y) by weight,
RYYRNWYNYYRWY (sequence ID number 8) peptide comprises 23.1% Arg (R), 15.4% Asn (N), 15.4% Trp (W), 46.2% Tyr (Y) by weight,
YYLYRNNWLIQLR (sequence ID number 9) peptide comprises 15.4% Arg (R), 15.4% Asn (N), 7.7% Gln (Q), 7.7% Ile (1), 23.1% Leu (L), 7.7% Trp (W), 23.1% Tyr (Y) by weight, and
RLLREWLNWTIQR (sequence ID number 10) peptide comprises 23.1% Arg (R), 7.7% Asn (N), 7.7% Gln (Q), 7.7% glutamic acid (Glu) (E), 7.7% Ile (1), 23.1% Leu (L), 7.7% Thr (T), 15.4% Trp (W) by weight.

The aim of the invention is to obtain sequence ID number 1, sequence ID number 2, sequence ID number 3, sequence ID number 4, sequence ID number 5, sequence ID number 6, sequence ID number 7, sequence ID number 8, sequence ID number 9 and sequence ID number 10 peptide sequences, which have the potential to show antibacterial activity when examined with computational methods based on artificial intelligence algorithms, providing both the elimination of bacteria and the inhibition of bacterial FadA protein-mediated tumorigenesis mechanisms.

Peptide development method of the invention comprises the process steps of:
Identifying the FadA interaction sequence on CDH1,
Detecting and analysing the active site of the FadA protein,
Analysing the ASANWTIQYND amino acid sequence that is the interaction site of the CDH1 protein with FadA, by the HPEPDOCK method,
Determining the interaction of the active site of the FadA protein with the reference ASANWTIQYND peptide sequence and determining the NWTIQ core interaction sequence,
Deriving 10000 peptide sequences with high binding energy using the interaction site,
Selecting 4973 peptide sequences with binding affinity<−14.0 kcal/mol using AutoDock CranckPep (ADCP) for local molecular docking analysis to derived peptide sequences,
Expanding the selected sequences with a genetic algorithm and performing local molecular docking analyses of the derived 37610 sequences,
Selecting the 100 sequences showing the highest binding after molecular docking analysis with 37610 sequences,
Evaluating the antimicrobial properties of 100 selected sequences using Random Forest (RF), Support Vector Machine (SVM) and Artificial Neural Network (ANN) model and selecting 56 peptide sequences with RF, SVM and ANN (probability)>0.5,
Performing global molecular docking analysis with HPEPDOCK to evaluate whether the selected sequences have interactions outside the active site on the FadA protein,
Re-performing FadA interaction analyses of selected sequences using ADCP, and
Selecting 10 peptide sequences by analysing the physical and chemical properties of peptides according to physiological conditions.

### DESCRIPTION OF THE DRAWINGS

FIG. 1a Mechanism of FadA Protein

FIG. 1b Inhibition of FadA Protein with sequence ID number 1-10 Peptides

FIG. 2a CDH1 FadA Protein Interaction Reference Peptide Sequence

FIG. 2b Representation of FadA Protein Active Site

FIG. 2c Analysis of Reference Peptide Sequence and FadA Active Site Interaction

FIG. 2d Generating Random Peptide from Reference Peptide, Propagation of Peptides Showing High Binding Energy with Genetic Algorithm and Molecular Docking of Propagated Peptides

FIG. 2e Selection of sequence ID number 1-10 Peptides Based on Evaluation of Physical, Chemical and Antimicrobial Properties of Peptides under Physiological Conditions

### DETAILED DESCRIPTION OF THE INVENTION

In the results of global molecular docking analysis of the peptides of the invention with anticancer and antimicrobial properties with the FadA protein of the reference peptide with the amino acid sequence ASANWTIQYND, after determining that the main interaction sequence of the sequence was NWTIQ, it was obtained by developing the first ten peptide sequences that were greater than the binding energy of the reference peptide to the FadA protein. With the invention, peptides were determined to prevent the interaction of the FadA protein released from F. nucleatum with E-cadherin, which has a carcinogenic effect, or to inhibit the microorganism with antimicrobial agents. Since it is difficult to use the ASANWTIQYND peptide sequence (reference peptide) in the literature for everyone, in silico studies have been carried out to derive more peptide sequences. The invention comprises peptide sequences with the highest binding energy to the FadA protein compared to the reference peptide. The relevant peptides were identified as TYYLRRTYKKQRH (sequence ID number 1), YRNWTIQRYRILR (sequence ID number 2), LRLIRRTIQVRTR (sequence ID number 3), RYYYLNWTIQRLR (sequence ID number 4), LRYRYNTIQYALR (sequence ID number 5), RRNWTWQRRLLRR (sequence ID number 6), YRRLRRLYRWYRY (sequence ID number 7), RYYRNWYNYYRWY (sequence ID number 8), YYLYRNNWLIQLR (sequence ID number 9) and RLLREWLNWTIQR (sequence ID number 10). In addition to allowing the development of treatment options for colorectal cancer, the invention also has a structure that can be modified for different diseases.

Sequence ID number 1 peptide comprises 23.1% arginine (Arg) (R) and 7.7% glutamine (Gln) (Q), 7.7% histidine (His) (H), 7.7% Leucine (Leu) (L), 15.4% lysine (Lys) (K), 15.4% threonine Thr (T), and 23.1% tyrosine Tyr (Y) by weight. sequence ID number 2 peptide comprises 30.8% Arg (R), 7.7% asparagine (Asn) (N), 7.7% Gln (Q), 15.4% isoleucine (Ile) (1), 7.7% Leu (L), 7.7% Thr (T), 7.7% tryptophan (Trp) (W), 15.4% Tyr (Y) by weight. sequence ID number 3 peptide comprises 38.5% Arg (R), 7.7% Gln (Q), 15.4% Ile (1), 15.4% Leu (L), 15.4% Thr (T), and 7.7% valine (Val) (V) by weight. sequence ID number 4 peptide comprises 23.1% Arg (R), 7.7% Asn (N), 7.7% Gln (Q), 7.7% Ile (1), 15.4% Leu (L), 7.7% Thr (T), 7.7% Trp (W), 23.1% Tyr (Y) by weight. sequence ID number 5 peptide comprises 7.7% alanine (Ala) (A), 23.1% Arg (R), 7.7% Asn (N), 7.7% Gln (Q), 7.7% Ile (1), 15.4% Leu (L), 7.7% Thr (T), 23.1% Tyr (Y) by weight. sequence ID number 6 peptide comprises 46.2% Arg (R), 7.7% Asn (N), 7.7% Gln (Q), 15.4% Leu (L), 7.7% Thr (T), 15.4% Trp (W) by weight. sequence ID number 7 peptide comprises 46.2% Arg (R), 15.4% Leu (L), 7.7% Trp (W), 30.8% Tyr (Y) by weight. sequence ID number 8 peptide comprises 23.1% Arg (R), 15.4% Asn (N), 15.4% Trp (W), 46.2% Tyr (Y) by weight. sequence ID number 9 peptide comprises 15.4% Arg (R), 15.4% Asn (N), 7.7% Gln (Q), 7.7% Ile (1), 23.1% Leu (L), 7.7% Trp (W), 23.1% Tyr (Y) by weight. sequence ID number 10 peptide comprises 23.1% Arg (R), 7.7% Asn (N), 7.7% Gln (Q), 7.7% glutamic acid (Glu) (E), 7.7% Ile (1), 23.1% Leu (L), 7.7% Thr (T), 15.4% Trp (W) by weight.

The peptides of the invention have flexible interaction ability and are biocompatible, allowing the activation or suppression of specific molecular pathways through protein interactions. Sequence ID number (1-10) has the ability to bind to FadA protein with high affinity. At the same time, sequence ID number (1-10) peptide sequences have the potential to show antibacterial activity when examined with computational methods based on artificial intelligence algorithms. In this way, both the elimination of bacteria and the inhibition of bacterial FadA protein-mediated tumorigenesis mechanisms are achieved.

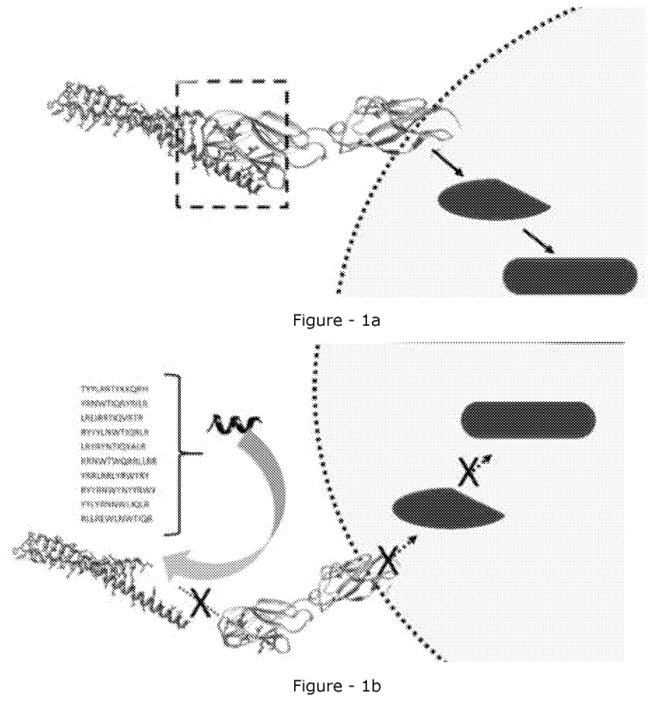

In FIG. 1a, FadA protein (1) interacts with E-cadherin, CDH1, (3) through its binding sites (2), leading to the production of oncogenes and tumorigenesis through β-catenin signalling within the cell. Therefore, preventing the interaction of the FadA protein released from the Fusobacterium nucleatum (F. Nucleatum) bacterium with E-cadherin or the production of molecules to inhibit the microorganism with antimicrobial agents seems to be an appropriate approach to inhibit tumorigenesis. To prevent the interactions of the FadA protein (1) with E-cadherin (3), the details of which are shown in FIG. 1b, it is observed that the use of ten peptide sequences (sequence ID number (1-10)) (4) designed as a result of analyses in molecular docking techniques can stimulate the abnormal β-catenin signalling pathway through E-cadherin and inhibit increased oncogene production.

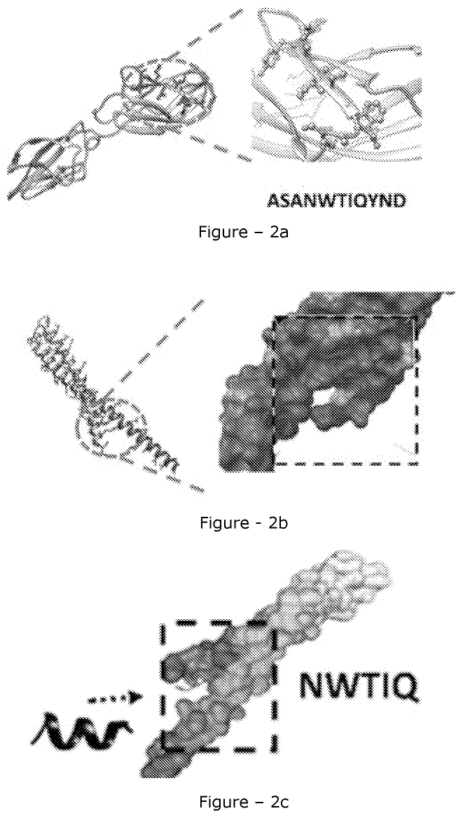

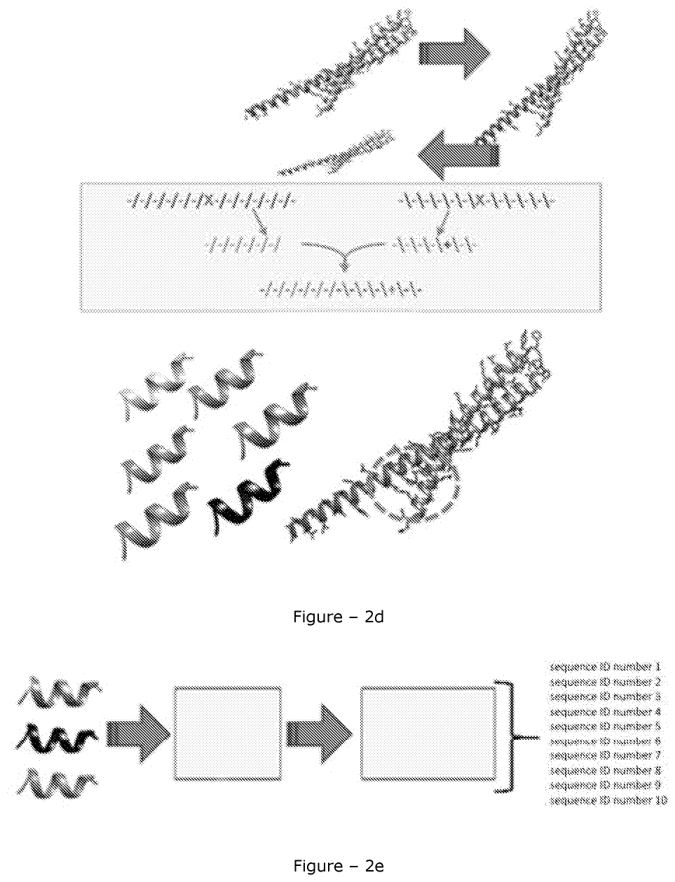

(FIG. 2a) The region that interacts with the FadA protein was found and extracted from the CDH1 protein sequence. For analysis, the crystal structure of the CDH1 protein was obtained from the ProteinDataBank (PDB accession number 7STZ) database in “.pdb” format. The obtained data file was examined in the context of the data in the literature and the position of the “ASANWTIQYND” sequence in the protein structure, which was used as a template in the further stages, was examined. Chimera (version 1.16) was used during the examinations. To determine the active site of the FadA protein, the AutoGrid Flexible Receptors (AGFR version 1.2) AutoSuite (version 1.1) program, which creates affinity maps of the binding sites of biomolecules, was used. (FIG. 2b) To determine the active site of the FadA protein, the AutoGrid Flexible Receptors (AGFR version 1.2) AutoSuite (version 1.1) program, which creates affinity maps of the binding sites of biomolecules, was used. The crystal structure of the FadA protein was obtained from the ProteinDataBank (PDB accession number 3ETW) database in “.pdb” format. The resulting data file was processed into “.pdbqt” format and provided as an input file to the AutoSuite program to find binding sites. A binding section consisting of 73 points was obtained in the part of the FadA protein that exhibited the best binding profile, and the affinity mapping parameters of the region were compiled as a “.trg” file. (FIG. 2c) In the results of global molecular docking analysis of the FadA protein and the reference ASANWTIQYND peptide sequence, it is determined that the main interaction sequence of the sequence was NWTIQ. (FIG. 2d) 10000 random sequences were generated to screen for sequences that would show higher affinity than the sequence of the region that interacts with the FadA protein of CDH1. Peptide sequences with good binding energy observed in local molecular docking analyses performed with these sequences were re-derived by random mutations and crossover with the genetic algorithm we developed. The resulting 37610 peptides were subjected to local molecular docking at the binding site of the FadA protein and the 100 peptides showing the highest binding were selected. (FIG. 2e) 100 peptides exhibiting high binding energy against the FadA protein were examined with tools that calculate the probability of antimicrobial peptides based on machine learning methods, and 56 peptides were found to have antimicrobial potential. To evaluate the profiles of peptides under physiological conditions, isoelectric point, water solubility and half-life parameters were evaluated with online tools. As a result, sequence ID number 1-10 peptide sequences were obtained.

Computational methods were used in the design of tumorigenesis inhibitor and antibacterial peptide, and the process steps were applied respectively. Identification of the FadA protein interaction sequence on CDH1; E-cadherin (extracellular), (CDH1) is a protein structure composed of five cadherin repeats. Tumorigenesis development is observed as a result of the regulation of oncogenes within the cell by stimulation of CDH1 through the ASANWTIQYND sequence (FIG. 2a). Flexible peptide structures that exhibit competitive behaviour with CDH1 to prevent E-cadherin from being subjected to abnormal cellular signalling are suitable mediators for inhibiting tumorigenesis. Detection of FadA protein active site; FadA protein is a biomolecule released from F. nucleatum that initiates CDH1-mediated increased oncogenic signals in human cells (FIG. 2b). In order to inhibit the activity of the protein in the human cell, formulations that will exhibit higher binding affinity than the CDH1 interaction site are suitable for tumorigenesis inhibition. Therefore, firstly, the binding properties of the reference ASANWTIQYND peptide with the FadA protein were examined. Analysis of interactions of the CDH1 reference peptide section (ASANWTIQYND) with the FadA protein; The peptide sequence extracted from the interaction site of the CDH1 protein with FadA was first subjected to flexible molecular docking analysis with the HPEPDOCK method. (HPEPDOCK: a web server for blind peptide-protein docking based on a hierarchical algorithm) HPEPDOCK analysis processes peptide conformational information as a bulk, without providing a specific binding site, and then models its semi-flexible docking to the input receptor. The program first predicts the secondary structures of the peptides given as a sequence and prepares 1000 conformations of each peptide, thus it works under cluster threshold 1 condition with a box size of 8.0 A, which can be described as semi-flexible. This global molecular docking analysis allows analysing both possible off-target bindings of the ‘ASANWTIQYND’ peptide with the FadA protein and important amino acids in the peptide sequence. When the obtained poses and bindings were examined, it was seen that the interaction of the FadA protein and the ASANWTIQYND reference peptide sequence mostly consisted of the NWTIQ motif.

Derivation of high binding energy peptide sequences using the NWTIQ motif template; It is known that the amino acids leucine (Leu, L), arginine (Arg, R) and tyrosine (Tyr, Y) are especially effective in the intra-interactions of the FadA-FadA protein. Therefore, while creating peptide derivatives, random peptide sets were prepared as follows with three different approaches; (FIG. 2d)
2000 peptides, 13 amino acids long, randomly selected from all amino acids,
5000 peptides with a random sequence of 8 amino acids from LYR amino acids, in which the NWTIQ motif [asparagine (Asn, N), tryptophan (Trp, W), threonine (Thr, T), isoleucine (Ile, 1) and glutamine (Gln, Q)] is randomly positioned,
3000 peptides with a random sequence of 8 amino acids from all amino acids and in which the NWTIQ motif is randomly positioned.

Local molecular docking analysis of randomly derived peptide sequences; the obtained affinity mapping parameters binding site file (“.trg”) and the obtained 10,000 random sequences were run with the AutoDock CrankPep (ADCP version 1.0) program with the top 10 scanning parameters in 500,000 steps. The ADCP program positions the peptide sequences provided as input files in the affinity maps defined in the local binding regions, predicts the binding energies (kcal/mol) and produces “.pdb” files showing the best 10 poses. Accordingly, 4973 peptide sequences showing the best affinity (binding affinity<−14 kcal/mol) were selected. (FIG. 2d) AutoDock CrankPep (ADCP) is a docking engine specialised for docking peptides. It combines filed protein folding technology with efficient representation of a rigid receptor as affinity grids to fold the peptide in the context of the energy landscape created by the receptor. A Monte-Carlo search is used to fold the peptide while simultaneously optimising the interaction between the peptide and the receptor molecule, yielding docked peptides. The program can provide peptides in PDB files or as 3D structures from a sequence of sequences. It has been shown to successfully translocate peptides up to 20 amino acids in length.

Expansion of high affinity random sequences with a genetic algorithm and local molecular docking analysis of the sequences; The peptide sequences showing the best binding, selected according to the ADCP results, were amplified by a genetic algorithm and mutation principle written in the R program. Briefly, the function splits the sequence of 13 amino acids into two parts, 6 and 7, creating two children, one of which is mutated with one of the 20 amino acids. In the function, the number of children is designed as a user-defined argument, and all but 1 of the expected number of children carries mutation. In this work step, 32637 new sequences produced from the 4973 best binding peptides selected in the 4^{th} work package were analysed under the same conditions as ADCP (top 10 scans in 500000 steps) (FIG. 2d).

Selection of 100 peptide sequences exhibiting the highest binding affinity and evaluation of their antimicrobial activity; the 37610 peptides for which molecular docking analysis was performed were sorted according to their binding affinity and the 100 sequences showing the best binding energy (the most negative) were selected. These peptide sequences were evaluated for their antimicrobial properties using the CAMPR4 (Collection of Anti-Microbial Peptides R4) database in “.fa” file format. CAMPR4 allows analysing the AMP properties of peptide sequences given as input files, with Random Forest (RF), Support Vector Machine (SVM) and Artificial Neural Networks (ANN) models established with all existing peptides (patented, experimentally proven or natural). During the study, peptides that met with the “amp_probability>0.5” condition were compiled in all of these models, which were run with 100 selected peptides. (FIG. 2e) As a result of AMP prediction, it was observed that 56 out of 100 peptides met with the >0.5 condition in all algorithms (RF, SVM and ANN).

Reanalysing peptide:FadA interactions by global molecular docking analysis; since it provides the opportunity to perform global scanning, the peptides obtained in the previous steps in the HPEPDOCK program were scanned without giving the binding region in order to determine whether there was an interaction outside the binding region. In this way, the possible interactions of flexible peptide structures with the FadA protein were analysed with both local (ADCP) and global (HPEPDOCK: a web server for blind peptide-protein docking based on a hierarchical algorithm) molecular docking programs. (FIG. 2e) Evaluation of the physical and chemical properties of peptides according to physiological conditions; ProtParam (https://web.expasy.org/protparam/) was used to evaluate peptides according to physiological conditions.

TABLE 1
Peptides and their properties that may show tumorigenesis inhibitor and antimicrobial
activity after molecular docking and antimicrobial activity analysis

| | | ADCP | | | | | | Half- | |
|---|---|---|---|---|---|---|---|---|---|
| | | Binding | HPEPDOCK | | | | | Life in | |
| | | Affinity | Binding | | | | | Mammal | Water |
| Identifier | Sequence | (kcal/mol) | Score | RF | SVM | ANN | pI | (hours) | Solubility |
| sequence ID | TYYLRRTYKKQRH | −25.7 | −206.113 | 0.89 | 0.96 | 0.87 | 10.55 | 7.2 | Good |
| number 1 | | | | | | | | | |
| sequence ID | YRNWTIQRYRILR | −25.6 | −199.936 | 0.83 | 0.93 | 0.89 | 11.55 | 2.8 | Good |
| number 2 | | | | | | | | | |
| sequence ID | LRLIRRTIQVRTR | −25.4 | −182.857 | 0.97 | 0.99 | 0.98 | 12.60 | 5.5 | Good |
| number 3 | | | | | | | | | |
| sequence ID | RYYYLNWTIQRLR | −24.8 | −194.645 | 0.62 | 0.53 | 0.66 | 10.27 | 1.0 | Bad |
| number 4 | | | | | | | | | |
| sequence ID | LRYRYNTIQYALR | −24.7 | −198.668 | 0.68 | 0.69 | 0.69 | 10.27 | 5.5 | Bad |
| number 5 | | | | | | | | | |
| sequence ID | RRNWTWQRRLLRR | −24.5 | −195.181 | 0.93 | 0.99 | 0.99 | 12.70 | 1.0 | Good |
| number 6 | | | | | | | | | |
| sequence ID | YRRLRRLYRWYRY | −24.1 | −209.674 | 0.88 | 0.96 | 0.97 | 11.38 | 2.8 | Good |
| number 7 | | | | | | | | | |
| sequence ID | RYYRNWYNYYRWY | −23.8 | −210.622 | 0.75 | 0.69 | 0.67 | 9.70 | 1.0 | Bad |
| number 8 | | | | | | | | | |
| sequence ID | YYLYRNNWLIQLR | −23.7 | −199.101 | 0.58 | 0.64 | 0.84 | 9.70 | 2.8 | Bad |
| number 9 | | | | | | | | | |
| sequence ID | RLLREWLNWTIQR | −23.6 | −174.089 | 0.86 | 0.94 | 0.93 | 11.70 | 1.0 | Good |
| number 10 | | | | | | | | | |
| | ASANWTIQYND * | −11.6 | −168.021 | 0.43 | 0.04 | 0.07 | 3.80 | 4.4 | Bad |

(* Reference peptide)

The method of developing peptide sequence with anticancer and antimicrobial properties, comprising the process steps of:
a. Identifying the FadA interaction sequence on CDH1,
b. Detecting the active site of the FadA protein,
c. Analysing the interaction site of CDH1 protein with FadA,
d. Generation of a random peptide sequence from the sequence by detecting the peptide sequence with high binding energy using the interaction site,
e. Performing local molecular docking analysis on derived peptide sequences,
f. Expanding the selected sequences with genetic algorithm and mutation,
g. Performing local molecular docking analyses of the expanded sequences and selecting the sequences according to their binding affinities,
h. Selecting the peptide sequence according to antimicrobial peptide analysis using the Random Forest (RF), Support Vector Machine (SVM) and Artificial Neural Network (ANN) model of the selected sequences,
i. Performing global and local molecular docking analysis of selected sequences, and
j. Selecting peptide sequences by analyzing the physical and chemical properties of peptides according to physiological conditions.

The interaction sequence detected in step “a” is ASANWTIQYND (reference peptide). The interaction site analysis performed in step “c” is the HPEPDOCK method. The interaction site in step “d” is the NWTIQ sequence. Again, the peptide sequences derived in step “d” are 10000; 2000 of them are randomly selected 13 amino acid long peptide sets, 5000 of them are 13 amino acid long peptide sets formed from LYR amino acids with a length of 8 amino acids and in which the 5-amino acid NWTIQ sequence is randomly positioned, and 3000 of them are 13 amino acid long peptide sets randomly generated from all natural amino acids with a length of 8 amino acids and in which the NWTIQ sequence of 5 amino acids is randomly positioned. Step “e” comprises the selection of 4973 peptide sequences by analysis with AutoDock CranckPep (ADCP). Again, the “binding affinity of the peptides selected in step “e” is <−14 kcal/mol”. There are 32637 peptide sequences replicated in step “f”. The analysis method in step “g” is AutoDock CranckPep (ADCP). In addition, there are 100 peptides selected with ADCP in step “g”. In step “h”, the antimicrobial activity measurement method is collection of anti-microbial peptides R4 (CAMPR4). In addition, the “amp_probability” (average match probability) value of the peptides selected in step “h” is >0.5. Again, in step “h”, the analysis methods are Random Forest (RF), Support Vector Machine (SVM) and Artificial Neural Network (ANN), and the RF, SVM, ANN values of the 56 selected peptides are >0.5. In step “i”, the sequence analysis method is HPEPDOCK and ADCP. In step “j”, the physiological evaluation is made with the ProtParam tool and the selected peptides are 10. Additionally, the peptide sequences selected in step “j” are TYYLRRTYKKQRH (sequence ID number 1) or YRNWTIQRYRILR (sequence ID number 2) or LRLIRRTIQVRTR (sequence ID number 3) or RYYYLNWTIQRLR (sequence ID number 4) or LRYRYNTIQYALR (sequence ID number 5) or RRNWTWQRRLLRR (sequence ID number 6) or YRRLRRLYRWYRY (sequence ID number 7) or RYYRNWYNYYRWY (sequence ID number 8) or YYLYRNNWLIQLR (sequence ID number 9) or RLLREWLNWTIQR (sequence ID number 10). The FadA binding energies defined in the local binding regions of the selected peptide sequences measured by ADCP are sequence ID number 1 −25.7 kcal/mol, sequence ID number 2 −25.6 kcal/mol, sequence ID number 3 −25.4 kcal/mol, sequence ID number 4 −24.8 kcal/mol, sequence ID number 5 −24.7 kcal/mol, sequence ID number 6 −24.5 kcal/mol, sequence ID number 7 −24.1 kcal/mol, sequence ID number 8 −23.8 kcal/mol, sequence ID number 9 −23.7 kcal/mol, and sequence ID number 10 −23.6 kcal/mol.

1. A peptide sequence with anticancer and antimicrobial properties comprising TYYLRRTYKKQRH (sequence ID number 1) or YRNWTIQRYRILR (sequence ID number 2) or LRLIRRTIQVRTR (sequence ID number 3) or RYYYLNWTIQRLR (sequence ID number 4) or LRYRYNTIQYALR (sequence ID number 5) or RRNWTWQRRLLRR (sequence ID number 6) or YRRLRRLYRWYRY (sequence ID number 7) or RYYRNWYNYYRWY (sequence ID number 8) or YYLYRNNWLIQLR (sequence ID number 9) or RLLREWLNWTIQR (sequence ID number 10) amino acid sequences

2. The peptide sequence with anticancer and antimicrobial properties according to claim 1 wherein the sequence ID number 1 peptide comprises 23.1% arginine (Arg) (R), 7.7% glutamine (Gin) (Q), 7.7% histidine (His) (H), 7.7% leucine (Leu) (L), 15.4% lysine (Lys) (K), 15.4% threonine Thr (T), 23.1% tyrosine Tyr (Y) by weight.

3. The peptide sequence with anticancer and antimicrobial properties according to claim 1 wherein the sequence ID number 2 peptide comprises 30.8% Arg (R), 7.7% asparagine (Asn) (N), 7.7% Gin (Q), 15.4% isoleucine (He) (I), 7.7% Leu (L), 7.7% Thr (T), 7.7% tryptophan (Trp) (W), 15.4% Tyr (Y) by weight.

4. The peptide sequence with anticancer and antimicrobial properties according to claim 1 wherein the sequence ID number s peptide comprises 38.5% Arg (R), 7.7% Gin (Q), 15.4% He (I), 15.4% Leu (L), 15.4% Thr (T), and 7.7% valine (Vai) (V) by weight.

5. The peptide sequence with anticancer and antimicrobial properties according to claim 1 wherein the sequence ID number 4 peptide comprises 23.1% Arg (R), 7.7% Asn (N), 7.7% Gin (Q), 7.7% He (I), 15.4% Leu (L), 7.7% Thr (T), 7.7% Trp (W), 23.1% Tyr (Y) by weight.

6. The peptide sequence with anticancer and antimicrobial properties according to claim 1 wherein the sequence ID number 5 peptide comprises 7.7% alanine (Ala) (A), 23.1% Arg (R), 7.7% Asn (N), 7.7% Gin (Q), 7.7% He (I), 15.4% Leu (L), 7.7% Thr (T), 23.1% Tyr (Y) by weight.

7. The peptide sequence with anticancer and antimicrobial properties according to claim 1 wherein the sequence ID number 6 peptide comprises 46.2% Arg (R), 7.7% Asn (N), 7.7% Gin (Q), 15.4% Leu (L), 7.7% Thr (T), 15.4% Trp (W) by weight.

8. The peptide sequence with anticancer and antimicrobial properties according to claim 1 wherein the sequence ID number 7 peptide comprises 46.2% Arg (R), 15.4% Leu (L), 7.7% Trp (W), 30.8% Tyr (Y) by weight.

9. The peptide sequence with anticancer and antimicrobial properties according to claim 1 wherein the sequence ID number 8 peptide comprises 23.1% Arg (R), 15.4% Asn (N), 15.4% Trp (W), 46.2% Tyr (Y) by weight.

10. The peptide sequence with anticancer and antimicrobial properties according to claim 1 wherein the sequence ID number 9 peptide comprises 15.4% Arg (R), 15.4% Asn (N), 7.7% Gin (Q), 7.7% He (I), 23.1% Leu (L), 7.7% Trp (W), 23.1% Tyr (Y) by weight.

11. The peptide sequence with anticancer and antimicrobial properties according to claim 1 wherein the sequence ID number 10 peptide comprises 23.1% Arg (R), 7.7% Asn (N), 7.7% Gin (Q), 7.7% glutamic acid (Glu) (E), 7.7% He (I), 23.1% Leu (L), 7.7% Thr (T), 15.4% Trp (W) by weight.

12. A method of developing peptide sequence with anticancer and antimicrobial properties comprising the process steps of:
a. identifying the FadA interaction sequence on CDH1,
b. detecting the active site of the FadA protein,
c. analyzing the interaction site of CDH1 protein with FadA,
d. generating a random peptide sequence from the sequence by detecting the peptide sequence with high binding energy using the interaction site,
e. performing local molecular docking analysis on derived peptide sequences,
f. expanding the selected sequences with genetic algorithm and mutation,
g. performing local molecular docking analyses of the expanded sequences and selecting the sequences according to their binding affinities,
h. selecting the peptide sequence according to antimicrobial peptide analysis using the Random Forest (RF), Support Vector Machine (SVM) and Artificial Neural Network (ANN) model of the selected sequences, and
i. performing global and local molecular docking analysis of selected sequences, and j. Selecting peptide sequences by analyzing the physical and chemical properties of peptides according to physiological conditions.

13. The method of developing peptide sequence with anticancer and antimicrobial properties according to claim 12 wherein the interaction sequence detected in step “a” is ASANWTIQYND (reference peptide).

14. The method of developing peptide sequence with anticancer and antimicrobial properties according to claim 12 wherein the interaction site analysis performed in step “c” is the HPEPDOCK method.

15. The method of developing peptide sequence according to claim 12 with anticancer and antimicrobial properties of claim 1 wherein the interaction site in step “d” is the NWTIQ sequence.

16. The method of developing peptide sequence with anticancer and antimicrobial properties according to claim 12 wherein the peptide sequences derived in step “d” are 10000; 2000 of them are randomly selected 13 amino acid long peptide sets, 5000 of them are 13 amino acid long peptide sets formed from LYR amino acids with a length of 8 amino acids and in which the 5-amino acid NWTIQ sequence is randomly positioned, and 3000 of them are 13 amino acid long peptide sets randomly generated from all natural amino acids with a length of 8 amino acids and in which the NWTIQ sequence of 5 amino acids is randomly positioned.

17. The method of developing peptide sequence with anticancer and antimicrobial properties according to claim 12, comprising in step “e”, the process step of selecting 4973 peptide sequences by analysis with AutoDock CranckPep (ADCP).

18. The method of developing peptide sequence with anticancer and antimicrobial properties according to claim 12, wherein the “binding affinity of the peptides selected in step “e” is <−14 kcal/mol”.

19. The method of developing peptide sequence with anticancer and antimicrobial properties according to claim 12, wherein there are 32637 peptide sequences replicated in step “f”.

20. The method of developing peptide sequence with anticancer and antimicrobial properties according to claim 12, wherein the analysis method in step “g” is AutoDock CranckPep (ADCP).

21. The method of developing peptide sequence with anticancer and antimicrobial properties according to claim 12, wherein there are 100 peptides selected with ADCP in step “g”.

22. The method of developing peptide sequence with anticancer and antimicrobial properties according to claim 12, wherein in step “h”, the antimicrobial activity measurement method is collection of anti-microbial peptides R4 (CAMPR4).

23. The method of developing peptide sequence with anticancer and antimicrobial properties according to claim 21, wherein the “amp probability” (average match probability) value of the peptides selected in step “h” is >0.5.

24. The method of developing peptide sequence with anticancer and antimicrobial properties according to claim 22, wherein in step “h”, the analysis methods are Random Forest (RF), Support Vector Machine (SVM) and Artificial Neural Network (ANN), and the RF, SVM, ANN values of the 56 selected peptides are >0.5.

25. The method of developing peptide sequence with anticancer and antimicrobial properties according to claim 12, wherein in step “i”, the sequence analysis method is HPEPDOCK and ADCP.

26. The method of developing peptide sequence with anticancer and antimicrobial properties according to claim 12, wherein in step “j”, the physiological evaluation is made with the ProtParam tool and the selected peptides are 10.

27. The method of developing peptide sequence with anticancer and antimicrobial properties according to claim 12, wherein the peptide sequences selected in step “j” are TYYLRRTYKKQRH (sequence ID number 1) or YRNWTIQRYRILR (sequence ID number 2) or LRLIRRTIQVRTR (sequence ID number 3) or RYYYLNWTIQRLR (sequence ID number 4) or LRYRYNTIQYALR (sequence ID number 5) or RRNWTWQRRLLRR (sequence ID number 6) or YRRLRRLYRWYRY (sequence ID number 7) or RYYRNWYNYYRWY (sequence ID number 8) or YYLYRNNWLIQLR (sequence ID number 9) or RLLREWLNWTIQR (sequence ID number 10).

28. The method of developing peptide sequence with anticancer and antimicrobial properties according to claim 12, wherein the FadA binding energies defined in the local binding regions of the selected peptide sequences measured by ADCP are sequence ID number 1 −25.7 kcal/mol, sequence ID number 2 −25.6 kcal/mol, sequence ID number 3 −25.4 kcal/mol, sequence ID number 4 −24.8 kcal/mol, sequence ID number 5 −24.7 kcal/mol, sequence ID number 6 −24.5 kcal/mol, sequence ID number 7 −24.1 kcal/mol, sequence ID number 8 −23.8 kcal/mol, sequence ID number 9 −23.7 kcal/mol, and sequence ID number 10 −23.6 kcal/mol.
